(19) United States
(12) Patent Application Publication
(10) Pub. No.: US 2023/0380762 A1
(43) Pub. Date: Nov. 30, 2023

(54) TECHNIQUES FOR IMAGE-BASED EXAMINATION OF FLUID STATUS

(71) Applicants: FRESENIUS MEDICAL CARE HOLDINGS, INC., Waltham, MA (US); FRESENIUS MEDICAL CARE DEUTSCHLAND GMBH, Bad Homburg (DE)

(72) Inventors: Matthias Kuss, Bad Homburg (DE); Peter Kotanko, Waltham, MA (US)

(21) Appl. No.: 18/034,302

(22) PCT Filed: Oct. 28, 2021
(86) PCT No.: PCT/US2021/057027

§ 371 Date: Apr. 27, 2023

Related U.S. Application Data
(60) Provisional application No. 63/107,741, filed on Oct. 30, 2020.

(51) Int. Cl. A61B 5/00; G06T 7/00; G06T 7/11; A61B 5/0537

(52) U.S. Cl. CPC A61B 5/4878; G06T 7/0016; G06T 7/11; A61B 5/0077; A61B 5/4848; A61B 5/0537; G06T 2207/20081; G06T 2207/30201; G06T 2207/20084

(57) ABSTRACT

Systems and methods for the image-based determination of the fluid status of a patient are described. In one example, an apparatus may include at least one processor and a memory coupled to the at least one processor. The memory may include instructions that, when executed by the at least one processor, may cause the at least one processor to receive an image that may include at least one image of a portion of a patient, determine fluid status information for the patient by processing the image via a trained computational model, the trained computational model trained based on at least one training image of the patient and a corresponding physical measurement of fluid status, the fluid status information indicating a current fluid status of the patient, and determine a treatment recommendation for the patient based on the fluid status information. Other embodiments are described.

### CROSS-REFERENCE TO RELATED APPLICATIONS

This application is a National Stage of International Application Serial No. PCT/US2021/057027 filed Oct. 28, 2021, which claims the benefit to U.S. Provisional Application No. 63/107,741, filed Oct. 30, 2020, the entire contents of which are incorporated herein by reference in their entirety.

### FIELD

The disclosure generally relates to processes for examining physical characteristics of a patient based on images of at least one portion of the patient, and, more particularly, to image-based techniques for assessing a fluid status of a patient.

### BACKGROUND

Fluid status is a critical health indicator for many conditions, such as congestive heart failure and kidney disease. For example, it is important to monitor patients with end-stage renal disease (ESRD) for fluid overload, which is the accumulation of fluid in the body. ESRD patients may lose their ability to produce and release urine such that fluid intake cannot be excreted. This leads to an accumulation of fluid in the body. Most of this superfluous water is stored as extracellular fluid, which may be observable as swelling in the outer extremities. In fluid-overloaded patients, the interstitial volume increases, manifesting itself in tissue swelling and sometimes extreme edema.

Conventional techniques for determining fluid status typically involve direct physical measurement. Certain determinations may be based on indirect measurements such as patient weight or blood pressure (or other bio-parameters), which may be easily obtained, but are generally not accurate (for instance, patient weight changes may be based on other factors, such as food intake, besides changes in fluid status). Other determinations may be based on direct measurements, such as bioimpedance, which involves applying an electric current through a portion of the patient and determining a fluid status based on a resistance of the electric current through the portion. Although bioimpedance and other direct techniques may be more accurate than indirect measurements, they require expensive equipment, trained medical professionals, and more time to perform. Accordingly, direct techniques are typically only performed at long time intervals (for instance, once every six to eight weeks) and require the patient to visit a healthcare facility.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one embodiment, an apparatus may include at least one processor and a memory coupled to the at least one processor. The memory may include instructions that, when executed by the at least one processor, may cause the at least one processor to receive an image that may include at least one image of a portion of a patient, determine fluid status information for the patient by processing the image via a trained computational model, the trained computational model trained based on at least one training image of the patient and a corresponding physical measurement of fluid status, the fluid status information indicating a current fluid status of the patient, and determine a treatment recommendation for the patient based on the fluid status information.

In some embodiments of the apparatus, the portion of the patient may include at least one of a hand, a foot, and a face. In various embodiments of the apparatus, the physical measurement of fluid status may include at least one of a weight measurement, a blood pressure measurement, or a bioimpedance measurement. In exemplary embodiments of the apparatus, the physical measurement of fluid status may include a bioimpedance measurement.

In some embodiments of the apparatus, the instructions, when executed by the at least one processor, may cause the at least one processor to train the computational model using the at least one training image and the corresponding physical measurement. In various embodiments of the apparatus, the instructions, when executed by the at least one processor, may cause the at least one processor to preprocess the at least one training image via defining a region of interest in the at least one training image. In some embodiments of the apparatus, the region of interest may include an area of the at least one training image associated with determining fluid status.

In exemplary embodiments of the apparatus, the instructions, when executed by the at least one processor, may cause the at least one processor to associate the at least one training image with at least one physical measurement to indicate a fluid status for at least one training image. In various embodiments of the apparatus, the at least one training image may include a plurality of images taken during different fluid states. In some embodiments of the apparatus, the different fluid states may include pre-dialysis and post-dialysis.

In one embodiment, a method may include receiving an image comprising at least one image of a portion of a patient, determining fluid status information for the patient by processing the image via a trained computational model, the trained computational model trained based on at least one training image of the patient and a corresponding physical measurement of fluid status, the fluid status information indicating a current fluid status of the patient, and determining a treatment recommendation for the patient based on the fluid status information.

In some embodiments of the method, the portion of the patient may include at least one of a hand, a foot, and a face. In various embodiments of the method, the physical measurement of fluid status may include at least one of a weight measurement, a blood pressure measurement, a bioimpedance measurement. In exemplary embodiments of the method, the physical measurement of fluid status may include a bioimpedance measurement. In some embodiments of the method, the method may include training the computational model using the at least one training image and the corresponding physical measurement.

In various embodiments of the method, the method may include preprocessing the at least one training image via defining a region of interest in the at least one training image. In some embodiments of the method, the region of interest may include an area of the at least one training image associated with determining fluid status. In various embodiments of the method, the method may include associating the at least one training image with at least one physical measurement to indicate a fluid status for at least one training image.

In some embodiments of the method, the at least one training image may include a plurality of images taken during different fluid states. In exemplary embodiments of the method, the different fluid states may include pre-dialysis and post-dialysis.

### BRIEF DESCRIPTION OF THE DRAWINGS

By way of example, specific embodiments of the disclosed machine will now be described, with reference to the accompanying drawings, in which:

FIG. 1 illustrates a first exemplary operating environment in accordance with the present disclosure;

FIG. 2 illustrates a second exemplary operating environment in accordance with the present disclosure;

FIG. 3 illustrates a third exemplary operating environment in accordance with the present disclosure;

FIG. 4 illustrates a graph of physical measurement information in accordance with the present disclosure;

FIG. 5 depicts illustrative information for determining a necessary sensor size;

FIG. 6 depicts illustrative processed images of a portion of a patient in accordance with the present disclosure; and

FIG. 7 illustrates an embodiment of a computing architecture in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

The described technology is generally directed to processes, systems, and methods for the image-based determination of the fluid status of a patient. In various embodiments, a fluid status analysis process may include a training process or phase in which a computational model is trained using training images of one or more portions of a patient and/or patient population and physical measurements of the patient and/or patient population using a physical measurement method. The fluid status analysis process may include a monitoring process or phase in which at least one image of a portion of the patient may be input into the trained computational model to generate output indicating a fluid status of the patient.

The portion (or one or more portions) of the patient may be selected because it is typically subject to a measurable or otherwise discernable difference in one or more characteristics based on fluid status. Non-limiting examples of portions of a patient may include an extremity, an appendage, a hand, a foot, a face, a wrist, an ankle, a calf, a portion of skin, and/or the like. For example, a foot of a patient may swell, causing changes in certain physical characteristics of the foot, when the patient is in a fluid-overload condition. A fluid status may include any indicator or description of indicating the fluid status of a patient. Illustrative and non-restrictive fluid statuses may include fluid overload (hypervolemia), normal, low fluid level, hypovolemia, edema, variations thereof, stages thereof, combinations thereof, and/or the like.

During the training process, a patient or healthcare professional may take one or more training images of the portion of the patient using a personal image-capturing device or computing device (for example, a smartphone, a tablet computing device, and/or the like). Simultaneously or contemporaneously, physical measurements may be taken of the patient to determine the fluid status of the patient. Non-limiting examples of physical measurements may include bioimpedance, weight, blood pressure, area and/or volume (for instance, of a portion of the patient), and/or the like. In some embodiments, the physical measurement may include bioimpedance analysis (BIA). In various embodiments, the bioimpedance or BIA information may be obtained via a body composition monitor (BCM). For example, a patient may visit a healthcare facility for an intake process in which images of the patient's hands, feet, face, and/or the like are captured. During or near in time to the visit, a fluid status may be obtained via physical measurements, such as bioimpedance. In between bioimpedance measurements, fluid status may be inferred based on weight measurements. In this manner, the training images may be associated with actual, real-world fluid status determinations.

In some embodiments, the bioimpedance measurements may be used to determine or estimate a dry body weight of the patient. The dry body weight may be used in some embodiments as a comparing or calibrating value.

The training images and fluid status determinations may be used to train a computational model to recognize the fluid status of a patient based on information in the training images. For example, the computational model may operate to associate characteristics of the images with fluid statuses specified in the corresponding fluid status determinations made via physical measurements. In some embodiments, the computational model may be or may include one or more artificial intelligence (AI) models, machine learning (ML) models, deep learning (DL) models, neural network (such as a convolutional neural network (CNN) and/or variations thereof), combinations thereof, and/or the like.

During the monitoring process, a monitoring image is provided to a trained computational model to determine a fluid status of the patient. For example, a monitoring image of the patient may be captured by an imaging device, such as a digital camera. The monitoring image may be of a portion of the patient used in at least a portion of the training images. For example, if images of the patient's left hand were used during computational model training, the monitoring image may be an image of the left hand of the patient. The monitoring image may be processed by the fluid status analysis process. For example, the monitoring image may be provided as input to the trained computational model. The computational model may analyze the image to generate output in the form of a fluid status or fluid status estimation of the patient. In some embodiments, the fluid status or fluid status estimation may be output on a display of a computing device. In various embodiments, the computational model may analyze the image to generate a treatment recommendation based, at least on part, on the fluid status or fluid status estimation.

Accordingly, in some embodiments, a computational model may be trained on actual physical measurements of the patient (and/or an associated patient population) in combination with patient images. For example, a computational model may be trained on patient images of known fluid states calibrated based on physical measurements. A non-limiting example of a physical measurement may include bioimpedance. For instance, a series of images of a patient's hand may be taken and associated with fluid states confirmed via bioimpedance measurements. A computational model may be trained on the images and corresponding bioimpedance information. A subsequent image of the patient's hand may be provided to the computational model to determine a fluid status of the patient based on the image without requiring a physical measurement of the patient.

Although bioimpedance is used as an example in the present disclosure, embodiments are not so limited, as any type of physical measurement technique for determining fluid status may be used according to some embodiments.

The monitoring of fluid status is a critical aspect of treating patients with various conditions that may affect patient fluid levels, such as congestive heart failure and kidney disease, particularly end-stage renal disease (ESRD). The fluid status of a patient may indicate progression of the disease and/or a serious medical condition, particularly for patients undergoing dialysis, such as hemodialysis (HD) and peritoneal dialysis (PD) patients. Conventional techniques for determining patient fluid status generally involve physical measurements and/or evaluations performed on the patient. In one example, measurements of patient weight, blood pressure, and other physical characteristics may be obtained and used to estimate a fluid status. However, such indirect techniques for determining fluid status are not able to provide accurate results because, among other things, changes in patient physical characteristics may be caused by other factors besides patient fluid levels. In another example, serious cases of fluid overload may cause edema (swelling of the skin), which may be diagnosed via a physical examination of the patient in a clinical facility. Bioimpedance techniques for determining fluid levels have proven to generate accurate results. In general, bioimpedance involves applying electrodes to a portion of a patient, for instance, a calf, to generate an electric current through the portion of the patient. A fluid level may be determined based on a resistance of the electric current through the portion of the patient. Although accurate, bioimpedance requires costly equipment and must be performed by trained healthcare professionals within a healthcare facility, making this method costly and burdensome to patients. Accordingly, patients requiring fluid status monitoring may only have a bioimpedance evaluation performed over long time intervals, such as every one or two months. However, significant, serious fluid changes may occur for the patient between bioimpedance measurements which may not be detected. Accordingly, patients may benefit from an easy and automated method that is reliable and accurate for measuring fluid status.

Fluid status analysis processes according to some embodiments may provide multiple technological advantages and technical features over conventional systems. One non-limiting example of a technological advantage may include training a computational model using patient-specific or patient population specific physical measurements to determine a fluid status of a patient based on an image of a portion of the patient. In general, computational models, such as AI and/or ML models, may utilize data from large populations to generalize certain features characteristic for a condition of interest, such as detecting a certain object (for instance, a person or a car) within an image. Since physical appearances and the effects of fluid status may differ materially from patient-to-patient, a pure image-based AI/ML technique using conventional methods may not provide accurate results for determining fluid status, such as the onset of edema. Accordingly, some embodiments may overcome this problem by calibrating images of specific patients using their fluid status determined by a non-image based, physical measurement method, for instance bioimpedance analysis (BIA).

Accordingly, some embodiments may provide personalized calibration of images of patient's body parts using non-image related information reporting his/her fluid status. This information can be obtained via one or more physical measurements, such as visual examination of patient, bioimpedance, weight, blood pressure, and/or the like. Contemporaneous digital images may be taken of patient body parts particularly affected by fluid overload, such as the lower extremities, the hands, the face, the feet, and/or the like. These images may then be analyzed by AI/ML methods and correlated with the patient's fluid status to train a computational model to recognize fluid status of a patient as reported against physical measurements, such as BIA. By using a non-image based physical measurement method to determine or estimate patient fluid status, a ground truth may be determined for each patient regarding their fluid status. This ground truth may be used to label, calibrate, or otherwise process images for determining future patient fluid status based solely on images.

Accordingly, another non-limiting example of a technological advantage may include providing accurate and efficient processes for determining patient fluid status based on images of a portion of the patient. A further non-limiting example of a technological advantage may include providing a system for a patient to use an image of a body part to determine their fluid status, including at a remote location outside of a clinical facility. An additional non-limiting example of a technological advantage may include providing a process for determining the fluid status of a patient without requiring physical measurements of a patient (for instance, without requiring bioimpedance measurements, weight measurements, blood pressure measurements, and/or the like). Embodiments are not limited in this context.

Processes, techniques, methods, systems, and/or the like described in the present disclosure may be integrated into various practical applications. For example, the fluid status analysis process may be integrated into the practical application of training a computational model using training images and corresponding physical measurements so that future fluid status determinations may be based on monitoring images without requiring physical measurements. In another example, the fluid status analysis process may be integrated into the practical application of diagnosing a fluid status of a patient. In a further example, the fluid status analysis process may be integrated into the practical application of administering treatment to a patient, such as providing treatment options, recommendations, prescriptions, and/or the like based on patient information and a fluid status determination. For example, administration of a treatment may include determining a dosage of a drug, administering the dosage of a drug, determining a testing regimen, administering the testing regimen, determining a treatment regimen (such as a dialysis treatment regimen, parameters (for instance, ultrafiltration rate), or prescription), administering the treatment regimen, and/or the like. Embodiments are not limited in this context.

Additional technological advantages and integrations of embodiments into practical applications are described in and would be known to those of skill in the art in view of the present disclosure.

In some embodiments, the fluid status analysis process may be an internet-based, Software-as-a-Service (SaaS), and/or cloud-based platform that may be used by a patient or a healthcare team to monitor patients clinical care and can be used to provide expert third-party assessments, for example, as a subscription or other type of service to healthcare providers.

For example, the fluid status analysis process may operate in combination with a “patient portal” or other type of platform that a patient and healthcare team may use to exchange information. For instance, dialysis treatment centers mange in-home patients who receive treatment in their own home and in-center patients who receive treatment at a treatment center. The patients may be in various stages of renal disease, such as chronic kidney disease (CKD), end-stage renal disease (ESRD), and/or the like. In-home patients may take a image of a body part, using a smartphone or other personal computing device on a periodic basis (for instance, daily, weekly, monthly, and/or the like) or as necessary (for instance, based on the appearance and/or change of an abnormality). The image may be uploaded to a patient portal or other platform (e.g., cloud, distributed computing environment, “as-a-service” system, etc.) and routed to an analysis system operative to perform the fluid status analysis process according to some embodiments. Similarly, images of in-center patients may be taken by the patient and/or clinical staff and uploaded to the patient portal or similar system for access by the analysis system.

In some embodiments, patient images may be stored in a repository or other database, including, without limitation, a healthcare information system (HIS), electronic medical record (EMR) system, and/or the like. Images in the repository may be catalogued and indexed by patient including key clinical information, demographics, medical history, and/or the like to be processed by the analysis system at a patient level and/or a population level. Use of patient image information at a population level may require de-identification of protected health information (PHI) and/or other information capable of identifying a patient according to required regulations, protocols, and/or the like, such as Health Insurance Portability and Accountability Act of 1996 (HIPAA).

The analysis system may operate to compare a patient's most recent image to the patient's previous images to automatically spot trends and variances in the patient's fluid status using imaging analysis technology configured according to some embodiments. In some embodiments, the fluid status analysis system may provide an assessment or diagnosis and/or one or more treatment recommendations, which may be provided to a healthcare team.

The healthcare team may then review the recommendations and either accept, decline, or revise the intervention for the patient. Healthcare team interventions may be documented and stored in the repository on both a patient-level and a population-level so that they can be followed to monitor success rates and outcomes to provide further training data to computational models used according to some embodiments.

Accordingly, the fluid status analysis process may use computational models that may continuously learn and monitor outcomes and success rates and provide feedback, treatment recommendations, diagnoses, and/or the like to the clinical care team using patient-specific and/or population-level analytics. The population-level analytics may be segmented based on various properties, such as age, gender, disease state, national population, regional population, and/or the like.

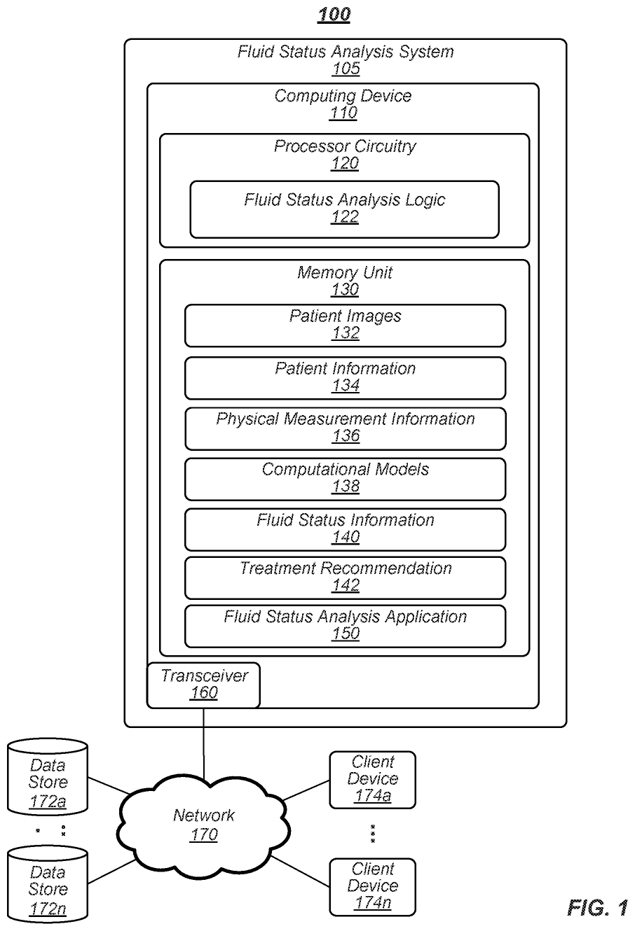

FIG. 1 illustrates an example of an operating environment 100 that may be representative of some embodiments. As shown in FIG. 1, operating environment 100 may include a fluid status analysis system 105. In various embodiments, fluid status analysis system 105 may include a computing device 110 communicatively coupled to network 170 via a transceiver 160. In some embodiments, computing device 110 may be a server computer or other type of computing device.

Computing device 110 may be configured to manage, among other things, operational aspects of a fluid status analysis process according to some embodiments. Although only one computing device 110 is depicted in FIG. 1, embodiments are not so limited. In various embodiments, the functions, operations, configurations, data storage functions, applications, logic, and/or the like described with respect to computing device 110 may be performed by and/or stored in one or more other computing devices (not shown), for example, coupled to computing device 110 via network 170 (for instance, one or more of client devices 174a-n). A single computing device 110 is depicted for illustrative purposes only to simplify the figure. Embodiments are not limited in this context.

Computing device 110 may include a processor circuitry that may include and/or may access various logics for performing processes according to some embodiments. For instance, processor circuitry 120 may include and/or may access a fluid status analysis logic 122. Processing circuitry 120, fluid status analysis logic 122, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 700. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, variations thereof, combinations of any of the foregoing, and/or the like.

Although fluid status analysis logic 122 is depicted in FIG. 1 as being within processor circuitry 120, embodiments are not so limited. For example, fluid status analysis logic 122 and/or any component thereof may be located within an accelerator, a processor core, an interface, an individual processor die, implemented entirely as a software application (for instance, a fluid status analysis application 150) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 130 may store various types of information and/or applications for a fluid status analysis process according to some embodiments. For example, memory unit 130 may store patient images 132, patient information 134, computational models 138, physical measurement information 136, fluid status information 140, treatment recommendations 142, and/or an fluid status analysis application 150. In some embodiments, some or all of patient images 132, patient information 134, physical measurement information 136, computational models 138, fluid status information 140, treatment recommendations 142, and/or an fluid status analysis application 150 may be stored in one or more data stores 172a-n accessible to computing device 110 via network 170. For example, one or more of data stores 172a-n may be or may include a HIS, an EMR system, a dialysis information system (DIS), a image archiving and communication system (PACS), a Centers for Medicare and Medicaid Services (CMS) database, U.S. Renal Data System (USRDS), a proprietary database, and/or the like.

Patient images 132 may include a digital or other electronic file that includes a image and/or video of a portion of a patient. The images may be stored as image files such as *.jpg, *.png, *.bmp, *.tif, and/or the like. In some embodiments, the images may be or may include video files such as *.mp3, *.mp4, *.avi, and/or the like. A patient, healthcare provider, caretaker, or other individual may capture the image using any capable device, such as a smartphone, tablet computing device, laptop computing device, personal computer (PC), camera, video camera, and/or the like. Patient images 132 may include training images and/or monitoring images. Training images may be used to train a computational model 138 during a training process of the fluid status analysis process. Monitoring images may be used to determine a fluid status of a patient via a trained computational model 138.

A user, such as the patient and/or healthcare professional, may send, transmit, upload, or otherwise provide patient images 132 to fluid status analysis system 105 via a client device 174a-n communicatively coupled to computing device 110 via network 170. For example, fluid status analysis application 150 may be or may include a website, internet interface, portal, or other network-based application that may facilitate uploading digital patient images 132 for storage in memory unit 130 and/or data stores 172a-n. In some embodiments, a patient client device 174a-n may operate a client application (for instance, a mobile application or “app”) operative to communicate with fluid status analysis application 150 for providing patient images 132. In some embodiments, a patient may upload digital patient images 132 via a patient portal of a dialysis clinic or other healthcare provider. Fluid status analysis application 150 may be communicatively coupled to the patient portal to receive images therefrom. Embodiments are not limited in this context.

In addition, a patient or healthcare provider may provide patient information 134 describing characteristics of the patient that may be relevant to determining fluid status. In general, patient information 134 may include any type of textual, audio, visual, and/or the like data outside of an patient image 132. For example, patient information 134 may include descriptions regarding pain, swelling, color, size, blood flow information, duration of a condition or characteristic, patient vitals, markings on skin (e.g., sock markings on swollen feet or ankles), geometry of body parts, and/or the like. In various embodiments, patient information 134 may be associated with one or more patient images 132, for example, as metadata, related within one or more medical record entries, and/or the like. For instance, fluid status analysis application 150 may create a record for an patient image 132 that includes or refers to associated patient information 134. In this manner, fluid status analysis application 150 may access information describing and/or providing context to an patient image 132.

In some embodiments, fluid status analysis application 150 may use one or more computational models 138 to analyze patient images 132 and/or patient information 134 to determine fluid status information 140 and/or treatment recommendations 142. Non-limiting examples of computational models 138 may include an ML model, an AI model, a neural network (NN), an artificial neural network (ANN), a convolutional neural network (CNN), a deep learning (DL) network, a deep neural network (DNN), a recurrent neural network (RNNs), a random forest algorithm, combinations thereof, variations thereof, and/or the like. Embodiments are not limited in this context. For example, a CNN may be used to analyze patient images 132 in which patient images 132 (or, more particularly, image files) are the input and fluid status information 140 (for instance, normal, fluid overload, etc.) and/or treatment recommendations 142 may be the output.

In various embodiments, fluid status analysis application 150 may use different computational models 138 for different portions of the fluid status analysis process. For example, an image-analysis computational model may be used to process patient images 132. In another example, a treatment recommendation computational model may be used to process patient information 134 and/or fluid status information 140 to generate a treatment recommendation 142. In some embodiments, one computational model 136 may be used for analyzing patient images 132, patient information 134, physical measurement information 136, and/or fluid status information 140 to determine a treatment recommendation 142. Embodiments are not limited in this context.

In various embodiments, physical measurement information 136 may include a fluid status determined via a physical measurement of the patient, such as via weight measurements, blood pressure measurements, bioimpedance measurements, and/or the like. For example, physical measurement information 136 may include a fluid status of normal as determined via BIA.

Fluid status analysis logic 122 may operate to perform a training process to train a computational model 138 using training images of patient images 132 and corresponding physical measurement information. Fluid status analysis logic 122 may operate to perform a monitoring process to determine fluid status information 140 of the patient by providing a monitoring image of the patient images 132 to a trained computational model 138. The trained computational model 138 may operate to generate fluid status information 140 indicating a fluid status of the patient based on the monitoring image.

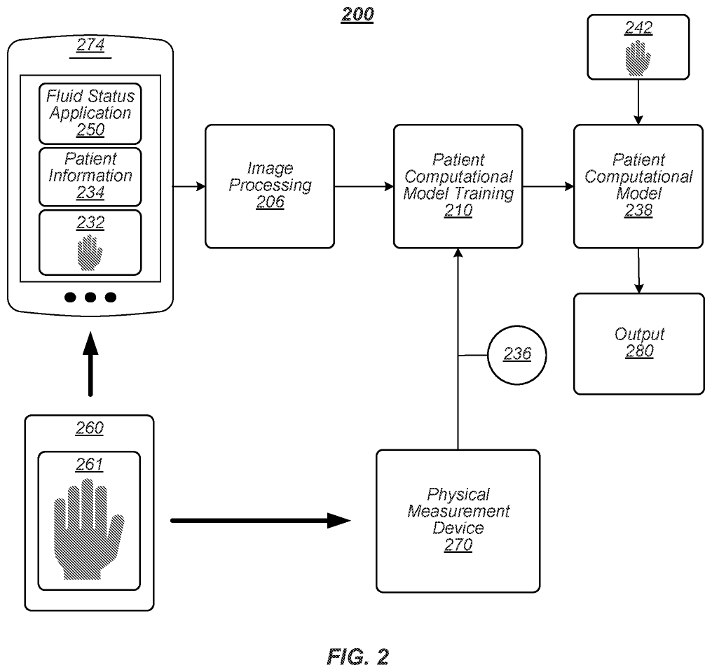

FIG. 2 illustrates an example of an operating environment 200 that may be representative of some embodiments. As shown in FIG. 2, operating environment 200 may include a physical measurement device, such as a BIA device or system. Physical measurement device 270 may operate to measure one or more physical characteristics of a patient 260 to determine a fluid status 236. A computing device 274 may capture a training image 232 of a portion 261 of patient 260. In some embodiments, training image 232 may include a plurality of images depicting different angles, orientations, sides, and/or the like of portion 261. For example, training image 232 may include multiple images of a hand of patient taken at different orientations. In some embodiments, image 232 may be of a plurality of portions 261 of patient 260 (for instance, hands, feet, face, etc.). In various embodiments, images 232 may be captured under different fluid states. For example, for a dialysis patent, images 232 may be captured both pre- and post-dialysis, during normal fluid status, during fluid overload, during low fluid conditions, and/or the like.

In various embodiments, at least a portion of images 232 may be undergo image processing 206. For example, in exemplary embodiments, images may be preprocessed to define the quadrant or region of interest (ROI) of the most applicable area in the image (e.g., foot, ankle, hand, portions thereof, etc.) and labeled with fluid status 236 determined by physical measurement. In some embodiments, a ROI may be a region determined to be associated with showing or otherwise indicating fluid status, such as edges of hands, fingers, feet, and/or the like. This processing step may create the “ground truth” for training computational model 238 for use in comparison of future images to determine fluid status of patient 260. In some embodiments, images 232 may include or may be used to generate 3D images.

In some embodiments, computing device 274 may be configured to access or determine patient information 134, such as patient identification information, patient physical characteristics, fluid status symptoms (for instance, swelling, presence of rashes or hives, joint pain, etc.), and/or the like. In various embodiments, computing device 274 may execute a client fluid status application 250 to facilitate the generation and/or management of images 232, patient information 234, and/or fluid status information 236. Images 232, patient information 234, and/or fluid status information 236 may be provided to a patient computational model training 210 logic to generate a trained computational model 238 using various DL techniques, such as pattern recognition, random forest, NN, etc.). Trained computational model 238 may be able to predict a fluid status of patient, such as a fluid deviation from a baseline for each patient based on a new image. For example, computational model 238 may be able to run an algorithm on a new image taken by patient 260 and compare it with a baseline (or estimated baseline) to estimate the fluid status of patient 260.

In some embodiments, at least a portion of images 232 and/or fluid status information 236 used to train computational model 238 may be generated from a patient population. In some embodiments, at least a portion of the training or development of computational model 238 may be based on patient population data. For example, a data collection phase may include taking periodic (e.g., weekly) measurements of patients (e.g., 10 patient, 25 patients, or more patients), such as weight and bioimpedance, and images of a body part (e.g., hand) in multiple angles both pre-dialysis and post-dialysis. The patient population information may be used to train computational model 238 to configure computational model 238 to be able to determine fluid status based on images. In a second or patient-specific training phase, images 232 and fluid status 236 may be specific to patient 260 to train computational model 238 specifically on patient 260 characteristics.

Monitoring images 242 may be provided to the trained computational model 238. For example, a patient may capture an image of a hand using a smartphone and the image provided to a fluid status analysis platform or application. Computational model 238 may analyze image 242 to generate an output 280, such as a fluid status (e.g., normal, fluid overload, and/or the like) and/or a treatment recommendation.

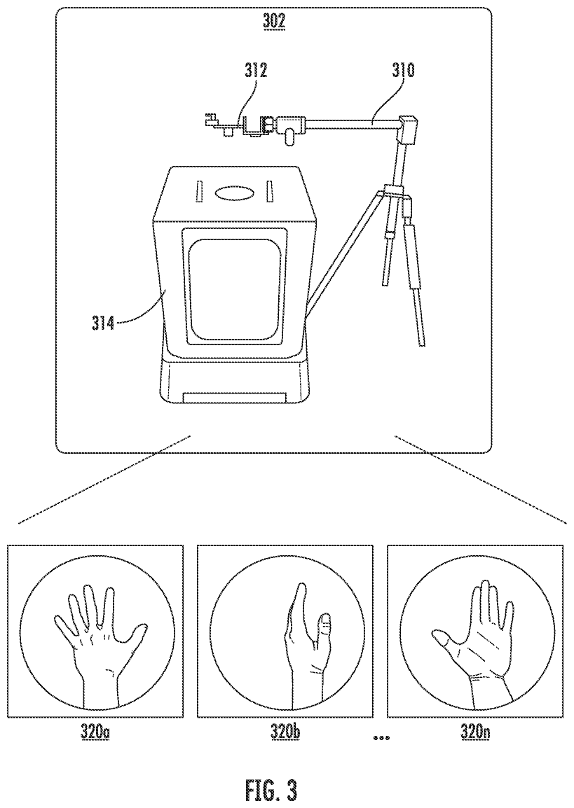

FIG. 3 illustrates an example of an operating environment 300 that may be representative of some embodiments. As shown in FIG. 3, operating environment 300 may include a mobile device 312 having a camera attached to a tripod 310, with the camera placed over a light box 314. In some embodiments, images 320a-n of a portion of a patient may be captured by placing the portion within light box 314. For example, for an HD or PD patient, an illustrative and non-restrictive procedure may include taking multiple images of a hand, such as a dorsal angle 320a, a side angle 320b, and a palmar angle 320n before and after dialysis treatment.

In some embodiments, various other sensors in addition to or in place of a camera may be used to generate images. For example, alternative imaging techniques may include, without limitation, infrared imaging, ultraviolet imaging, and other imaging techniques may be used to generate images. In various embodiments, images 320a-n may be used to generate three-dimensional (3D) models. For example, image Z-stacks may be used to cut 3D images into pieces and produce a 3D image afterwards. The alternative images and 3D models may be used to train computational models and/or to determine fluid status via a trained computational model according to some embodiments.

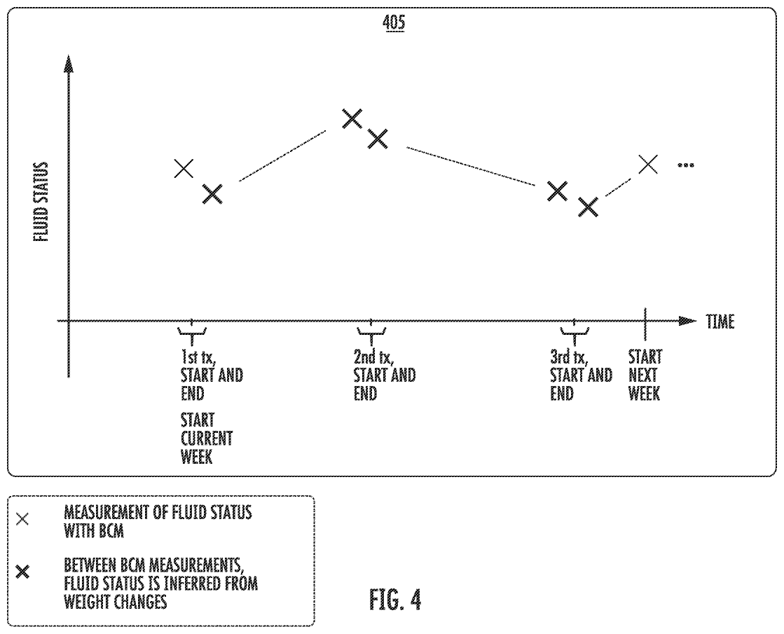

FIG. 4 illustrates calibration information for a fluid status analysis process according to some embodiments. As shown in graph 405 of FIG. 4, physical measurements may be taken over a period of time. For example, a BCM measurement and weight measurement may be taken at the start of a first dialysis treatment during a first week of measurement. Additional measurements may be taken during the first week, repeating when the second week begins. This information may be associated with patient images for training a computational model according to some embodiments.

In some embodiments, changes in fluid status may be reflected in extracellular water, which may be manifested in extremities, such as the hands, feet, and face. The distribution of extracellular water (or other fluids influencing fluid status) may be different for each patient (e.g., one patient may show more indications of fluid overload in the feet, while another patient may show more visual evidence of fluid overload in the face). Accordingly, personalized, trained computational models for determining patient fluid status according to some embodiments may be effective and accurate, compared to conventional techniques and/or non-personalized processes.

In some embodiments, fluid status (or changes in fluid status) may be based on changes in volume of an extremity, such as a hand, a foot, the face, and/or the like. In some embodiments, a correlation may be made between the area of an extremity (e.g., a hand) and the volume. Accordingly, in some embodiments, a change in the area of an extremity (e.g., a hand) may be used to determine or estimate a change in a volume (a total volume or an extracellular volume) of the hand (for instance, which may be used to determine a fluid status change or estimate). In exemplary embodiments, a change in the area (and therefore, the volume) of the extremity may be determined to indicate a change in fluid status and, in some embodiments, the change in fluid status may be quantified. In some embodiments, the volume of an extremity, such as a hand, may be determined based on a water-displacement technique (i.e., place hand in known volume of water in a container, determine difference in volume of water to be the volume of the hand; compare previous water-displacement measurements to determine differences in volume of hand or other extremity). In various embodiments, extremity volume may be used because the measurement focus may be on the blood overload status of the extremity.

### Use Case Example

Mean fluid shifts in dialysis patients in arms were 11.98+-6.76% measured before and after performing of hemodialysis. For dialysis patients, 2.5 to 3.5 liters of ultrafiltrate is common. Thus, this difference of fluid accumulation in the body should at least be measurable. While the objective is to be able to determine the fluid status quantitatively, this measurement minimum is necessary to at least tell, if the patient is having an immense fluid overload. Calculating the difference in swelling can be simplified by the assumption that fluid accumulates directly under the skin's surface. Several formulas for calculating the Body Surface Area (BSA) of human body exist. For example, the formula of Mosteller, given by the following Equation (1) with Hₜ as the body height and Wₜ as the patient's weight:

$$B S A ( {m}^{2} ) = \sqrt{\frac{{H}_{t} ( cm ) \cdot {W}_{t} ( kg )}{3600}}$$

Assuming an average dialysis patient with a height of about 1.73 m and a weight of 80 kg, the amount of swelling can be calculated over the surface. As the average patient has a BSA of about 1.96 m 2, the gain to be measured can be determined at about 1.5 mm. To eliminate certain variables, the image capture process may be standardized (see, for example, FIG. 3). For example, to ensure a good lightning, the images are taken in a photo light box, for instance, with 2200 lumen and 40 cm height. Putting a human hand into the box leads to a distance from camera to hand of about 35 cm. Based on conventional user technology, such as smart phones, to capture images, and assumption of usable camera resolution may be between 12-13 MP with a focal length of 3.6-4.25 mm, and a pixel size of about 1.2-1.4 μm. With this information, sensor resolution may be provided by the following Equation (2):

FOV*focal length=sensor size*working distance.

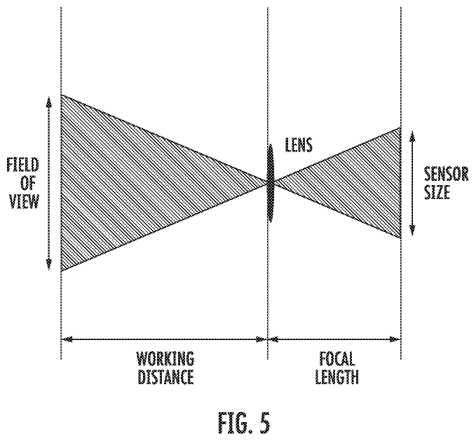

FIG. 5 depicts illustrative information for determining a necessary sensor size.

To determine the necessary resolution for digitalization of features, the calculation of Nyquist criterion shown in Equation (3) may be used. Given a FOV of about 20 cm (based on the size of an average hand) and a resolution of about 3000 pixels leads to a smallest detectable feature of 0.13 mm. Assuming that the swelling can even make a difference of about 1.5 mm, the change should be easily detectable. Regarding Abbe diffraction limit for smallest features that are still detectable as distinct objects, a minimum lens angle needs to be met, to capture all secondary maxima of scattered light. Assuming a straight lightning without conductor the formula for the minimum limit of object size can be calculated with Equation (4) based on the wavelength of light λ, the refractive index n as well as the half-angle of lens θ. In embodiments in which the image is captured without involving a specific medium, it can be assumed that the refraction index is 1 (air). The angle itself can be calculated out of sensor size and focal length on basis of the angular aperture which is defined as shown in Equation (5) with diameter of aperture D. The calculations lead to a lens angle of about 13.2°. Given a wavelength of 450-650 nm of LED white, the Abbe diffraction limit may be set to 380-549 nm.

$$Abbe limit d = \frac{λ}{2 \cdot n sin θ} = \frac{λ}{2 NA} \qquad Equation ( 3 )$$

$$Numerical aperture N A = n sin θ \qquad Equation ( 4 )$$

$$Angular aperture θ = arc tan ( \frac{D / 2}{focal length} ) \qquad Equation ( 5 )$$

There may be multiple factors potentially influencing the recognition of swelling or other characteristics of fluid status. For a computational model, such as a model using a DL algorithm, all or partially all changes in hand appearance may play a role in the classification of images. Besides jewelry and nail color, skin irritations and wounds may be detected or recognized by the process. A list of influencing factors and associated values are included in the following Table 1:

TABLE 1
Disturbance and/or influencing factors on image analysis

| | | | |
|---|---|---|---|
| Background | Pre-position of hand | Rheumatism | Dilation of hand geometry |
| Nail colour | Movement of patient | Obesity | Wounds, Rash, skin irritations, sunburn |
| Washing hands | Blood pressure | Oedema | Equilibrium time of fluid status |
| Age spots | Squeezing factors | Jewellery | Cardiac insufficiency |
| Lighting | Resolution camera | Band aid | Temperature outside/season |
| Amputations | Temperature of clinic | Hairiness | Vascular Access problems |
| Skin colour | Distance to camera | Crinkles | Liver spots |

Certain factors, such as background and lighting may be eliminated, for example, through the usage of a photo-light box. Using this standardized environment, in combination with setting the distance between camera and hand (or other body part) to the determined range, depending on how high the patient holds his hand into the box, may alleviate certain influencing factors. Other influencing factors like the temperature around or the preposition of the hand that can provoke swelling may be determined to determine their overall impact on the measurement. Nail color, wounds, sun burn, liver spots, wrinkles, jewelry (for instance, by changing body part shape and/or causing artificial swelling) and other factors may play a role, for example, when deep learning is applied and the algorithm is searching for changes in hand appearance automatically. However, such factors may generally be neglected when only measuring the shape changes of outer hand (or other body part) edges.

In addition to the influence factors, the mode of image capture may have an influence on fluid status determinations. While vascular access problems can lead to asymmetric swelling in the extremity closer to the access, this mismeasurement can be avoided by taking images of both hands. Following this process, edema may also be reduced or eliminated as an influencing. When using a BCM to measure the fluid status, the patient may be required to remain in a static (half sitting) position for a time period, such as about 15 minutes. In this manner, fluid is distributed over the body and no fluid shifts are influencing the measurements. As the swelling of the hand (or other body part) may also depend on temperature of the environment and the position, further mismeasurement can be minimized by keeping the patient in the measuring position for the time period (for instance, about 15 minutes). The patient may be measured and/or images captured multiple times (for example, two times). In some embodiments, the BCM measurements are captured at the same or substantially the same time as capturing the images, without moving the patient. In various embodiments, a BCM or the bioimpedance measurement system with flat electrodes to lay the hands (or other body part) may be used.

The timing of image capture and/or physical measurements may also be another influencing factor. For example, for HD patients, there is typically a dialysis break on weekends, such that the biggest difference of fluid status should be measured before last dialysis on Friday in comparison with fluid status before measurement on Monday.

In various embodiments, calibration objects (such as a coin or ruler) may be used to calibrate the size of the imaged objects (e.g., hands, feet, etc.) to pixels in the corresponding digital image. In some embodiments, the training images and/or monitoring images may be standardized or correlated to facilitate accuracy. For example, the training images and/or monitoring images may be taken in the same positions (e.g., angle, hand open/closed, orientation, etc.), lighting conditions, and/or the like.

In some embodiments, images may be pre-processed to remove noise and other unwanted influencing factors. For example, noise may be removed and filters (such as texture filters) may be used to highlight the edges of the hand (or other body part), for instance, as with adaptive threshold. In some embodiments, a binary segmentation of the image may be built up. In a next step, the image may be further cleaned (for instance, water shedding) and by shrinking lines to a single pixel line. After the preprocessing, the measurement can take place. For example, performance of image-wide measurements, per-feature, pixel-by-pixel or relative segmentation measurements may be conducted.

In one embodiment, for example, only swelling of the hand (or other body part) may be measured. Therefore, it may be sufficient to show the hand outlines and measure the distance between the left and right sides of the image. In some embodiments, at least one tool that may be used for image analysis is OpenCV. In the beginning of preprocessing, the image may be converted to grayscale. The next step may include converting the image into a binary format, such as adaptive binary. In some embodiments, the adaptive binary format may be achieved via a Gaussian adaptive binary function. In some embodiments, additional bilateral blurring effects may be applied to eliminate noise but keep the edges clean, followed by an Otsu binarization, which automatically tries to find the most fitting threshold t for a given bimodal image. The Otsu algorithm does that by minimization of weighted within-class variances with weights q in I bins of the histogram given by the relation shown in the following Equation (6):

$${σ}_{w}^{2} ( t ) = {q}_{1} ( t ) \cdot {σ}_{1}^{2} ( t ) + {q}_{2} ( t ) \cdot {σ}_{2}^{2} ( t )$$

$$where$$

$${q}_{1} ( t ) = {\sum }_{i = 1}^{t} P ( i ) \& {q}_{1} ( t ) = {\sum }_{i = t + 1}^{I} P ( i )$$

$${μ}_{1} ( t ) {\sum }_{i = 1}^{t} \frac{i \cdot P ( i )}{{q}_{1} ( t )} \& {μ}_{2} ( t ) = {\sum }_{i = t + 1}^{I} \frac{i \cdot P ( i )}{{q}_{2} ( t )}$$

$${σ}_{1}^{2} ( t ) = {\sum }_{i = 1}^{t} {[ i - {μ}_{1} ( t ) ]}^{2} \frac{P ( i )}{{q}_{1} ( t )} \& {σ}_{2}^{2} ( t ) = {\sum }_{i = t + 1}^{I} {[ i - {μ}_{1} ( t ) ]}^{2} \frac{P ( i )}{{q}_{2} ( t )}$$

In various embodiments, additional processing may include use of low pass filters, application of erosion and dilation (to get rid of black noise by using the closing function), and Canny Edge (for example, to delete noise remaining on the outer edge of the hand itself).

The following Equation (7) shows the calculation of different bioimpedance measurements that may be used to physically determine fluid status according to some embodiments:

$$Arm ( A ) {RI}_{A} = \frac{arm length ( {cm}^{2} )}{{R}_{A} ( Ω )} Leg ( L ) {RI}_{L} = \frac{leg length ( {cm}^{2} )}{{R}_{L} ( Ω )}$$

$$Trunk ( T ) {RI}_{T} = \frac{trunk height ( {cm}^{2} )}{{R}_{T} ( Ω )}$$

$$Full body ( total ) {RI}_{total} = \frac{total body height ( {cm}^{2} )}{{R}_{R} A ( Ω ) + {R}_{L} A ( Ω ) + {R}_{T} ( Ω ) + {R}_{R} L ( Ω ) + {R}_{L} L ( Ω )}$$

$$R A = right arm , L A = left arm , R L = right leg , L L = left leg$$

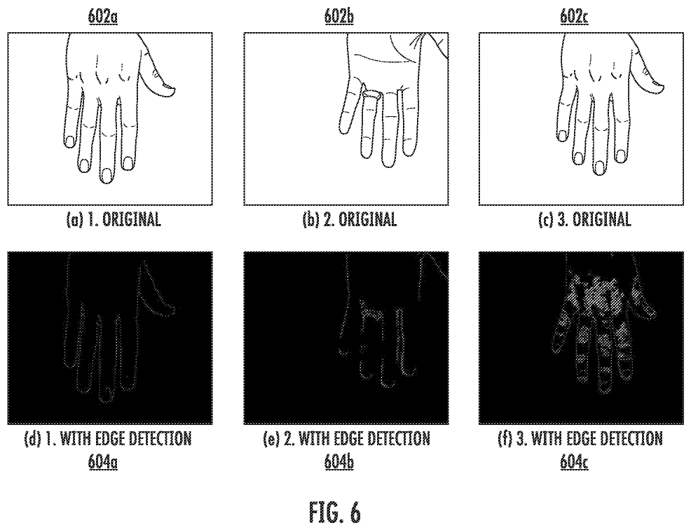

Different feature detection algorithms can be applied to find the most useful features. For example, different feature detection algorithms are tested in terms of their performance for detection of features in hand images. FIG. 6 depicts illustrative raw or original images of hands 602a-n and corresponding processed images 604a-n, for example, with edge detection.

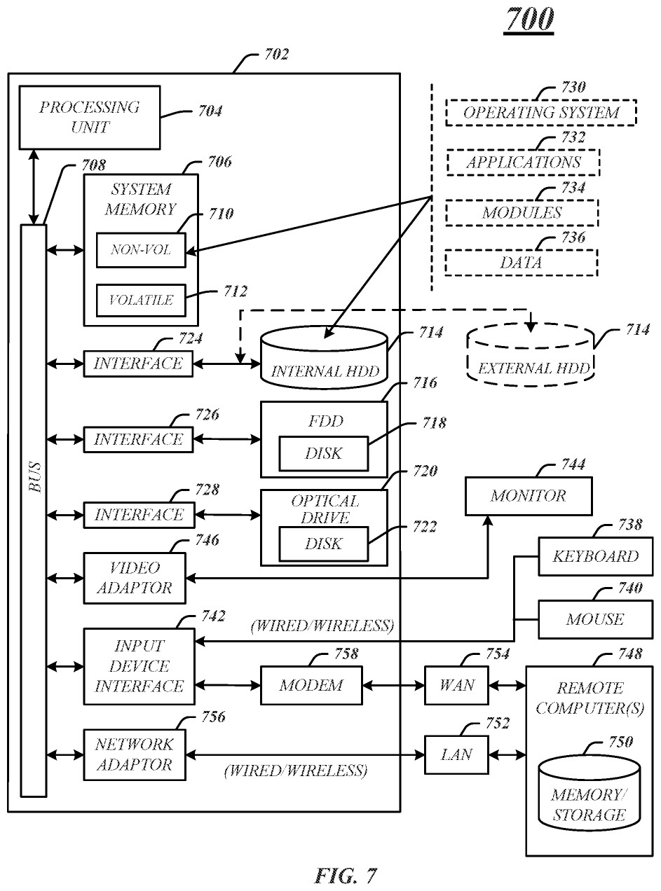

FIG. 7 illustrates an embodiment of an exemplary computing architecture 700 suitable for implementing various embodiments as previously described. In various embodiments, the computing architecture 700 may comprise or be implemented as part of an electronic device. In some embodiments, the computing architecture 700 may be representative, for example, of computing device 110. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 700. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 700 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 700.

As shown in FIG. 7, the computing architecture 700 comprises a processing unit 704, a system memory 706 and a system bus 708. The processing unit 704 may be a commercially available processor and may include dual microprocessors, multi-core processors, and other multi-processor architectures.

The system bus 708 provides an interface for system components including, but not limited to, the system memory 706 to the processing unit 704. The system bus 708 can be any of several types of bus structure that may further interconnect to a memory bus (with or without a memory controller), a peripheral bus, and a local bus using any of a variety of commercially available bus architectures. Interface adapters may connect to the system bus 708 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 706 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 7, the system memory 706 can include non-volatile memory 710 and/or volatile memory 712. A basic input/output system (BIOS) can be stored in the non-volatile memory 710.

The computer 702 may include various types of computer-readable storage media in the form of one or more lower speed memory units, including an internal (or external) hard disk drive (HDD) 714, a magnetic floppy disk drive (FDD) 716 to read from or write to a removable magnetic disk 711, and an optical disk drive 720 to read from or write to a removable optical disk 722 (e.g., a CD-ROM or DVD). The HDD 714, FDD 716 and optical disk drive 720 can be connected to the system bus 708 by a HDD interface 724, an FDD interface 726 and an optical drive interface 728, respectively. The HDD interface 724 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 710, 712, including an operating system 730, one or more application programs 732, other program modules 734, and program data 736. In one embodiment, the one or more application programs 732, other program modules 734, and program data 736 can include, for example, the various applications and/or components of computing device 110.

A user can enter commands and information into the computer 702 through one or more wired/wireless input devices, for example, a keyboard 738 and a pointing device, such as a mouse 740. These and other input devices are often connected to the processing unit 704 through an input device interface 742 that is coupled to the system bus 708, but can be connected by other interfaces.

A monitor 744 or other type of display device is also connected to the system bus 708 via an interface, such as a video adaptor 746. The monitor 744 may be internal or external to the computer 702. In addition to the monitor 744, a computer typically includes other peripheral output devices, such as speakers, printers, and so forth.

The computer 702 may operate in a networked environment using logical connections via wired and/or wireless communications to one or more remote computers, such as a remote computer 748. The remote computer 748 can be a workstation, a server computer, a router, a personal computer, portable computer, microprocessor-based entertainment appliance, a peer device or other common network node, and typically includes many or all of the elements described relative to the computer 702, although, for purposes of brevity, only a memory/storage device 750 is illustrated. The logical connections depicted include wired/wireless connectivity to a local area network (LAN) 752 and/or larger networks, for example, a wide area network (WAN) 754. Such LAN and WAN networking environments are commonplace in offices and companies, and facilitate enterprise-wide computer networks, such as intranets, all of which may connect to a global communications network, for example, the Internet.

The computer 702 is operable to communicate with wired and wireless devices or entities using the IEEE 802 family of standards, such as wireless devices operatively disposed in wireless communication (e.g., IEEE 802.17 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. An apparatus, comprising:
at least one processor;
a memory coupled to the at least one processor, the memory comprising instructions that, when executed by the at least one processor, cause the at least one processor to:
receive an image comprising at least one image of a portion of a patient,
determine fluid status information for the patient by processing the image via a trained computational model, the trained computational model trained based on at least one training image of the patient and a corresponding physical measurement of fluid status, the fluid status information indicating a current fluid status of the patient, and
determine a treatment recommendation for the patient based on the fluid status information.

2. The apparatus of claim 1, the portion of the patient comprising at least one of a hand, a foot, and a face.

3. The apparatus of claim 1, the physical measurement of fluid status comprising at least one of a weight measurement, a blood pressure measurement, or a bioimpedance measurement.

4. The apparatus of claim 1, the physical measurement of fluid status comprising a bioimpedance measurement.

5. The apparatus of claim 5, the logic to train the computational model using the at least one training image and the corresponding physical measurement.

6. The apparatus of claim 5, the logic to preprocess the at least one training image via defining a region of interest in the at least one training image.

7. The apparatus of claim 6, the region of interest comprising an area of the at least one training image associated with determining fluid status.

8. The apparatus of claim 5, the logic to associate the at least one training image with at least one physical measurement to indicate a fluid status for at least one training image.

9. The apparatus of claim 5, the at least one training image comprising a plurality of images taken during different fluid states.

10. The apparatus of claim 9, the different fluid states comprising pre-dialysis and post-dialysis.

11. A method, comprising:
receiving an image comprising at least one image of a portion of a patient;
determining fluid status information for the patient by processing the image via a trained computational model, the trained computational model trained based on at least one training image of the patient and a corresponding physical measurement of fluid status, the fluid status information indicating a current fluid status of the patient; and
determining a treatment recommendation for the patient based on the fluid status information.

12. The method of claim 11, the portion of the patient comprising at least one of a hand, a foot, and a face.

13. The method of claim 11, the physical measurement of fluid status comprising at least one of a weight measurement, a blood pressure measurement, or a bioimpedance measurement.

14. The method of claim 11, the physical measurement of fluid status comprising a bioimpedance measurement.

15. The method of claim 15, comprising training the computational model using the at least one training image and the corresponding physical measurement.

16. The method of claim 15, comprising preprocessing the at least one training image via defining a region of interest in the at least one training image.

17. The method of claim 16, the region of interest comprising an area of the at least one training image associated with determining fluid status.

18. The method of claim 15, comprising associating the at least one training image with at least one physical measurement to indicate a fluid status for at least one training image.

19. The method of claim 15, the at least one training image comprising a plurality of images taken during different fluid states.

20. The method of claim 19, the different fluid states comprising pre-dialysis and post-dialysis.
